(19) United States
(12) Patent Application Publication
(10) Pub. No.: US 2003/0213314 A1
(43) Pub. Date: Nov. 20, 2003

(54) Biometric energy identification system

(76) Inventor: Stefaan De Schrijver, Newton, MA (US)

Correspondence Address: LAHIVE & COCKFIELD, 28 STATE STREET, BOSTON, MA, 02109, US

(21) Appl. No.: 10/299,982

(22) Filed: Nov. 19, 2002

Related U.S. Application Data
(63) Continuation of application No. PCT/US01/16056, filed on May 17, 2001.
(60) Provisional application No. 60/205,735, filed on May 19, 2000.

(51) Int. Cl. G01L 5/00; A61B 5/11; A61B 5/117

(52) U.S. Cl. 73/865.4; 702/179

(57) ABSTRACT

A writing instrument that is configured with sensors to measure biometric data including the friction forces, accelerations, and angles applied by the writing instrument to a writing surface during a writing event such as a signature. The mean energy dissipated by a valid signature owner during a signature event, is significantly lower than that of a forger or other falsifier. By recording and storing the mean minimal energy for registered, authorized signature owners, a signature energy database can be compiled. Registered authorized signature owners are positively identified by writing their signature with the biometric writing instrument that communicates the energy information to the energy database for verification. The energy database may be located at a remote location to provide signature verification when the signature requester and signature provider are not co-located.

### RELATED INFORMATION

The present application is a continuation of PCT patent application number PCT/US01/16056, filed on May 17, 2001, which claims priority to U.S. provisional patent application No. 60/205,735, filed on May 19, 2000, the entire contents both of which are hereby incorporated by reference.

### BACKGROUND OF THE INVENTION

1. Field of the Invention

The present invention relates generally to an apparatus and system for user identification and authentication, and more specifically to an apparatus and system that measures biometric data during a writing event.

2. Description of the Prior Art

Reliable and accurate identity verification is critical to merchants and consumers with the increasing demand for more convenient, electronic transactions. As electronic commerce increases, however, so does the opportunity for fraudulent transactions and security system breaches. Some systems have employed identity verification methods including signatures, personal identification numbers (PINs), digital images, and fingerprints, or combinations thereof, to decrease probability of false identification.

For example, the credit card industry uses signatures for positive identification, however there is not complete uniformity among this industry as some credit card issuers attempt to increase credit card security by imprinting digital images on the issued cards. Unfortunately, this lack of conformity within industries translates to even greater disparity amongst the technologies employed by various industries. This lack of conformity is expensive for the industries and can be frustrating and inconvenient for the consumer. Additionally, the lack of consistency amongst industries leads to consumer uncertainty regarding the effectiveness of certain merchant methods.

A signature is one authentication method used widely throughout many industries, with the signature providing authorization for credit card transactions, legal transactions, etc. The signature is therefore widely accepted by both consumers and merchants, but the problem is that a signature often requires the immediate presence of both parties. Additionally, signatures may be forged, with particular individuals able to forge signatures with relative ease.

There is currently no apparatus, system, or methodology to improve the signature authentication method, and allow for remote verification of signatures.

What is needed is an apparatus and system that increases signature authentication reliability, and provides remote signature authentication in a cost-efficient, predictable manner.

### SUMMARY OF THE INVENTION

The present invention is an apparatus and method that implements a writing instrument to measure the friction forces, accelerations, and angles as a user applies a writing instrument to a writing surface while writing a signature. The friction forces have a significantly lower mean energy when a signature is performed by the signature owner than by a forger or other falsifier under duress. The present invention measures and records the mean energy generated by a signature. The energy levels are compared to a database containing the mean minimal energy values for the signature owner. Positive identification is ensured when the particular signature energy is within an acceptable energy dissipation range of the database mean minimal energy value.

Other objects and advantages of the present invention will become more obvious hereinafter in the specification and drawings.

### BRIEF DESCRIPTION OF THE DRAWINGS

A more complete understanding of the invention and many of the attendant advantages thereto will be readily appreciated as the same becomes better understood by reference to the following detailed description when considered in conjunction with the accompanying drawing, wherein like reference numerals refer to like parts and wherein:

FIG. 1 presents a architectural diagram of the verification system components.

### DESCRIPTION OF THE ILLUSTRATED EMBODIMENTS

To provide an overall understanding of the invention, certain illustrative embodiments will now be described; however, it will be understood by one of ordinary skill in the art that the systems described herein can be adapted and modified to provide systems for other suitable applications and that other additions and modifications can be made to the invention without departing from the scope hereof.

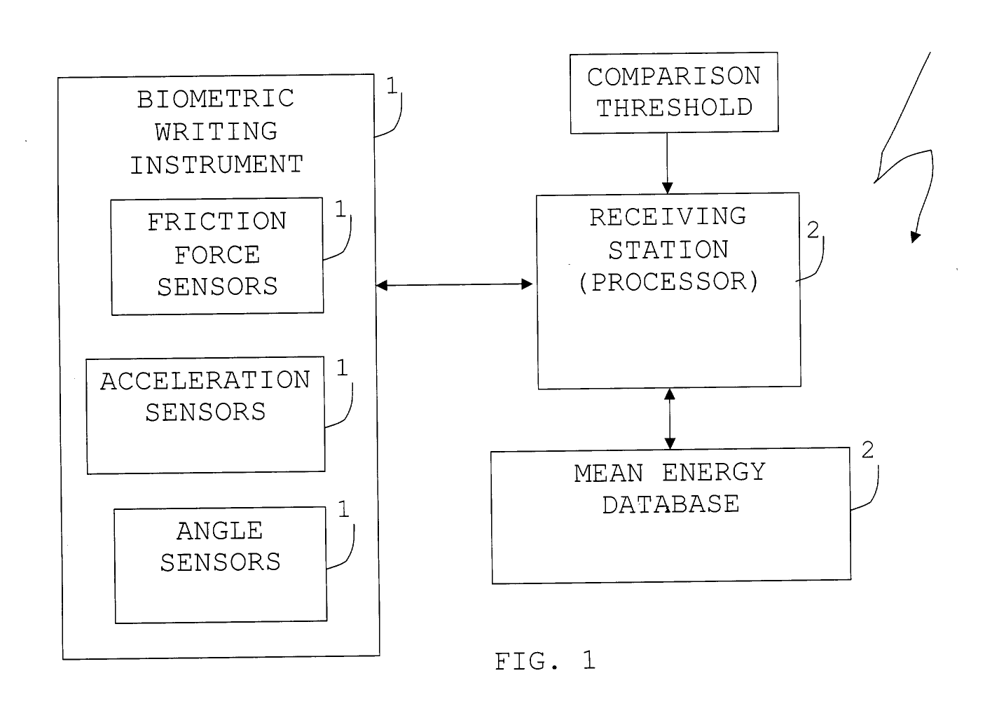

Referring now to FIG. 1, there is shown a block diagram of the system architecture 10 for a signature verification system. The writing instrument 12 in the illustrated system 10 is a pen that is instrumented to acquire biometric information during a writing event such as a signature, although those with ordinary skill in the art will recognize that the system can be applied to any writing event without departing from the scope of the invention. Although a pen shall be discussed, one skilled in the art will recognize that the invention herein is not limited to a pen, and the writing instrument can be a pencil or stylus. Additionally, it is not necessary that the writing instrument provide a visible mark on a particular surface, rather it is only necessary that the writing instrument contact some surface during a writing event. In the illustrated embodiment, the writing event shall be referred to as a signature event, although it is not necessary that the writing instrument user generate a signature, and any other marking may be utilized without departing from the invention herein.

As shown by FIG. 1, the illustrated embodiment writing instrument 12 includes sensors that allow the measurement of the mean energy during the signature event. In the illustrated embodiment, such sensors include at least one friction force sensor 14, at least one acceleration sensor 16, and at least one angle sensor 18. The aforementioned sensors are merely illustrative, and the invention is not limited to such sensors, but rather encompasses all sensors that, alone or in combination, measure the energy dissipation during a signature event.

In the FIG. 1 embodiment, friction force sensors 14 measure the friction force between the writing instrument 12 and the surface upon which the writing instrument is acting during the signature event, the friction force being measured in three orthogonal directions. Additionally, acceleration sensors 16 measure the acceleration of the writing instrument in three directions during the signature event. The FIG. 1 embodiment additionally includes three angle sensors 18 to measure the angles between the writing instrument and the writing surface.

The friction, acceleration, and angle measurements of the FIG. 1 system can be transmitted to a receiving station 20 with a user identifier. The communication link between the writing instrument 12 and the receiving station 20 can be wired or wireless, and the invention herein is not limited by the communication method or apparatus. The receiving station 20 can be a stand-alone system or a networked system, and in the illustrated embodiment, the receiving station is a personal computer. The invention herein is also not limited to the depicted receiving station apparatus, and can include any microprocessor controlled device that can perform the functions attributed to the receiving system 20 described herein. Accordingly, the receiving station 20 can be a SUN™ workstation, handheld, palmtop, laptop, telephone, personal digital assistant (PDA), etc., without departing from the scope of the invention.

In an embodiment, the user identifier is a personal identification number (PIN) entered by the user, however, the invention is not limited to the method or data that identifies the individual signature grantor of a particular signature event.

In an embodiment, the receiving station 20 processes the measured friction, acceleration, and angle information from a particular signature event to generate the energy dissipated during the signature event. The receiving station then retrieves data from the mean energy dissipation database 22 for the user identified by the PIN. The mean energy dissipation database can include mean energy values for registered users who are identified through respective PINs. The mean energy values can be derived by obtaining a representative number of signatures from the registered users using the biometric writing instrument 12, performing the receiving station processing on the representative signatures, and computing the average energy dissipation value for storage in the database 22. During subsequent user signature events wherein signature verification is desired, the mean energy dissipation can be retrieved from the database 22 and compared against the single event energy dissipation. In the illustrated system, if the comparison is not within a specified threshold 24, the signature is not authorized. In some embodiments, the threshold 24 can be variable and established by a system administrator, etc., depending upon the application.

The illustrated embodiment allows a variable comparison threshold 24 because different applications can desire different security levels. For example, more expensive transactions can desire a very strict comparison between the obtained signature and the database, while less costly transactions can allow a more liberal authorization criteria.

The verification results in the FIG. 1 embodiment can be displayed on a display in communications with the receiving station 20, although the invention is not so limited to such display of information. In an alternate embodiment, the writing instrument can include a display for indicating the verification results, or the verification results can be displayed on yet another device that is connected, using a wire or wireless communications connection, to the receiving device 20. Those with ordinary skill in the art will recognize that the invention herein is not limited to the communications protocol.

One advantage of the present invention over the prior art is that the present invention provides a system and method for using biometric energy to validate a writing sample or event.

What has thus been described is a writing instrument that is configured with sensors to measure biometric data including the friction forces, accelerations, and angles applied by the writing instrument to a writing surface during a writing event such as a signature. The mean energy dissipated by a valid signature owner during a signature event is significantly lower than that of a forger or other falsifier. By recording and storing the mean minimal energy for registered, authorized signature owners, a signature energy database can be compiled. Registered authorized signature owners are positively identified by writing their signature with the biometric writing instrument that communicates the energy information to the energy database for verification. The energy database may be located at a remote location to provide signature verification when the signature requester and signature provider are not co-located.

Although the present invention has been described relative to a specific embodiment thereof, it is not so limited. Obviously many modifications and variations of the present invention may become apparent in light of the above teachings. For example, the sensors to generate the energy dissipation measurements may be replaced by other sensors that similarly provide an energy dissipation calculation. The connections between the writing instrument, the receiving station, and the database may be achieved using well known communications techniques that include wired, contact, and wireless methods. The receiving station and database may be one structure, or separate structures. Similarly, the receiving station and processing functions may be separate. The method to identify the requester may include a name, PIN, or other process. The energy dissipation computation may be performed within a processor inside the writing instrument, wherein the energy value is passed to the receiving station; or, the receiving station may process the biometric signals to obtain the mean energy value.

Many additional changes in the details, materials, steps and arrangement of parts, herein described and illustrated to explain the nature of the invention, may be made by those skilled in the art within the principle and scope of the invention. Accordingly, it will be understood that the invention is not to be limited to the embodiments disclosed herein, may be practiced otherwise than specifically described, and is to be understood from the following claims, that are to be interpreted as broadly as allowed under the law.

What is claimed is:

1. A system for verifying signatures comprising a writing instrument, the writing instrument further comprising at least one sensor to measure energy dissipation during a signature event.

2. A system according to claim 1, wherein the writing instrument further comprises at least one friction force sensor to measure friction force between the writing instrument and a writing surface during the signature event.

3. A system according to claim 1, wherein the writing instrument further comprises at least one acceleration sensor to measure acceleration of the writing instrument during the signature event.

4. A system according to claim 1, wherein the writing instrument further comprises at least one angle sensor to measure angles between the writing instrument and the writing surface during the signature event.

5. A system according to claim 1, further comprising a receiving device to receive and process at least one of a friction, acceleration, and angle sensor measurement received during the signature event, the received measurements to be processed by the receiving device to generate an energy dissipation value.

6. A system according to claim 1, further comprising a database containing mean dissipation energy corresponding to signature events for authorized users.

7. A system according to claim 1, further comprising a module to communicate the measured friction, acceleration, and angle measurements to the receiving device.

8. A system according to claim 1, further comprising a PIN database.

9. A method for verifying signatures, comprising:
collecting data from sensors on a writing instrument during a writing instrument event;
specifying a claimed identity of the writing instrument user;
computing the mean energy dissipated during the writing instrument event; and,
comparing the mean energy dissipated during the writing instrument event, to historic mean energy data for the claimed identity.

10. A method according to claim 9, wherein collecting data further comprises acquiring data from at least one friction force sensor to measure friction force between the writing instrument and a writing surface.

11. A method according to claim 9, wherein collecting data further comprises acquiring data from at least one acceleration sensor to measure acceleration of the writing instrument during the writing instrument event.

12. A method according to claim 9, wherein collecting data further comprises acquiring data from at least one angle sensor to measure angles between the writing instrument and the writing surface during the writing instrument event.

13. A method according to claim 9, further comprising compiling a historical database of mean energy data wherein mean energy data is correlated with known identities.
